(19) United States
(12) Patent Application Publication
(10) Pub. No.: US 2018/0357553 A1
(43) Pub. Date: Dec. 13, 2018

(54) METHOD OF PREDICTING THE LEVEL OF PHYSICAL DEMAND REQUIRED OF AN INDIVIDUAL TO FACE A PATH, ESPECIALLY A BICYCLE TOURING ROUTE

(71) Applicants: Paolo MARESCA, Roma (IT); Marco GIOVACCHINI, Pietrasanta (IT)

(72) Inventors: Paolo MARESCA, Roma (IT); Marco GIOVACCHINI, Pietrasanta (IT); Stefano STRANO, Roma (IT)

(21) Appl. No.: 15/778,162

(22) PCT Filed: Nov. 24, 2016
(86) PCT No.: PCT/IB2016/057112

§ 371 Date: May 22, 2018

(30) Foreign Application Priority Data
Nov. 24, 2015 (IT) 102015000076223

(51) Int. Cl. G06N 5/04; G16H 50/30

(52) U.S. Cl. CPC G06N 5/048; G16H 50/30

(57) ABSTRACT

A method determines the level of physical demand required of an individual to face a given path, especially a bicycle-touring route. The method includes the steps of determining a physical profile of the individual, i.e. his/her ability to sustain a given effort during physical activity, determining the degree of difficulty of the path and comparing the physical profile with the degree of difficulty to determine the level of physical demand required of the individual. The method can be advantageously carried out by a program installed on a server with a Web interface, or by an application for a smart phone, a tablet or a PC.

### TECHNICAL FIELD

The present invention relates to a method of predicting the level of physical demand required of an individual to face a path.

Particularly, the invention relates to a method that can be implemented in a Web-application, or an application for a smart phone, a tablet, a PC or the like, which method can predict the level of physical demand required of an individual to face a given bicycle touring route, while ensuring well-being and safety in terms of fitness and health.

### PRIOR ART

The ability of an individual to complete a path with an adequate level of physical effort, using a bicycle is known to depend on multiple factors, which are substantially associated with the physical skills of the individual and the difficulties of the selected path.

Nevertheless, in the field of physical exercise for wellbeing and fitness purposes, no method exists that can provide objective, evidence-based assessment of the fitness of an individual to face a given path, particularly a bicycle touring route using a bicycle, under safe conditions and with a beneficial effect on fitness.

Furthermore, on the one hand, currently used methods of assessment of the functional skills of an individual are based on complex protocols that have been designed for competitive-level or performance-oriented physical training. On the other hand, the parameters that have been conventionally used to assess the difficulty of a path, such as length and elevation gain, which can be freely retrieved from Web map and navigation services are not related to each other and cannot be directly compared to the parameters for assessment of functional skills of an individual.

### DISCLOSURE OF THE INVENTION

Therefore, the object of the invention is to provide a user-friendly method that can provide objective assessment of the level of physical demand required of an individual to face a given path, especially a bicycle touring route, according to both his/her functional skills and the difficulties of the path.

A further object of the invention is to provide a method that allows a user to select the path that most suits his/her skills while ensuring safety and wellbeing.

Another object of the invention is to provide a method that can be implemented through a Web-application or a an application designed for a smart phone, a tablet, a PC or similar devices.

These objects are fulfilled by a method as defined in the annexed claims, which method substantially comprises a preliminary step of determining, on the one hand, the physical profile of an individual, i.e. his/her ability to sustain a given effort during physical activity, and on the other hand the degree of difficulty of given path, especially a bicycle touring route, and a later step of comparing said physical profile with said degree of difficulty to determine the level of physical demand required of the individual.

The method further includes the step of finding and comparing two synthetic indexes, representative of the physical profile of the individual and the degree of difficulty of the path respectively.

Namely, the synthetic index representative of the physical profile is determined by administering a questionnaire to the individual, e.g. concerning his/her characteristics and/or physical skills, lifestyle, etc.

The physical profile in the present method is based on biometric measurements, on behavioral information and on indirect assessment of the functional skills of the individual, derived from the perception of effort intensity when practicing sports. Such physical profile constitutes a predictive instrument that the present method considers prior to actual exercise by the individual, to reduce the risk of damages to the cardiovascular system caused by excessive muscular load during sports activity. The ultimate goal consists in allowing the individual to practice sports under safe conditions.

The synthetic index representative of the difficulty of the path is determined, for example, by accounting for the length of the path, the cumulative elevation gain, the maximum grade, etc.

The present method assesses the difficulties of the path based on cartographic data, before the individual starts it, by assigning scores to allow path selection.

Thus, the present method has the purpose of allowing the individual to autonomously plan his/her training schedule, and avoid unsuitable paths, by comparing two directly comparable synthetic indexes.

A method according to the present invention provides the advantage of providing an indication of the level of physical demand required, which will allow the individual to face the path under well-being and health safety conditions, while accounting for the functional skills of the individual that is going to start the path, as shown by tests carried out by a primary University institution.

The present invention provides the additional advantage of allowing the user to select the most suitable path for his/her skills.

Further advantages will result from the following detailed description of an exemplary embodiment of the method of the present invention.

### EMBODIMENTS OF THE INVENTION

According to the invention, in order to assess the level of physical demand required of an individual when starting a bicycle touring route, a first synthetic index F, representative of the physical profile of an individual—i.e. his/her ability to sustain a given effort during physical activity, and a second synthetic index P, representative of the difficulty of the desired path, are first determined.

Particularly, the first synthetic index F is determined according to
a) parameters representative of the performance capabilities of the individual;
b) parameters representative of the ability of the individual to adapt to physical activity;
c) health risk parameters for the individual.

These parameters are combined together, with given weights being assigned to each factor based on scientific evidence.

In practice, the first synthetic index F is assigned a numerical value based on the answers given by the individual to a questionnaire concerning, for instance:
1. Age
2. Sex
3. Body Mass Index, calculated from weight and height
4. Waist circumference
5. Heartbeat rate at rest
6. Smoking
7. Hours exercise per week
8. Type of sport being practiced
9. Effort intensity perceived during physical activity.

The second synthetic index (P) is in turn calculated from parameters representative of the potential energy expenditure (both total and per time unit) required, such parameters being combined together according to the total load and the point loads in path segments.

In practice, the second synthetic index P is assigned a numerical value based on the following factors, appropriately weighted:
1′. Total distance
2′. Cumulative elevation gain (i.e. the sum of the elevation gains of uphill slopes)
3′. Maximum grade %

According to an advantageous peculiarity of the present method, the two synthetic indexes assume values in the same scale and of the same order of magnitude.

In other words, the above two synthetic indexes F and P are designed to be directly comparable, which provides an apparent innovation as compared with the methods as used heretofore, and affords considerable advantages.

Once the two synthetic indexes have been determined, the level of physical demand required of the individual to face the given path can be obtained from the relationship I=f(F,P)

where f is a function that relates the indexes F and P to each other.

A possible advantageous function f is shown below.

Assuming that the index F may have values ranging from 0 to F_{MAX} (e.g. 33) and the index P may take values ranging from 0 to P_{MAX} (e.g. 40) and assuming I=P−F

| | LEVEL OF PHYSICAL DEMAND |
|---|---|
| | REQUIRED |
| I <− m | Very easy with respect to the profile |
| −m ≤ I <− n | Easy with respect to the profile |
| −n ≤ I ≤ n | In line with the profile |
| n > I ≤ m | Demanding with respect to the profile |
| I > m | Very demanding with respect to the |
| | profile |

Where m and n are integral numbers, preferably ranging from 1 to 10, and m>n, e.g. m=4 and n=2.

In typical use, the method is carried out on a server with a Web interface, or by an application for a smart phone, a tablet or a PC.

The users answers the questions to determine his/her profile and later selects a bicycle touring route through a graphics interface (such as the one provided by Google Maps service). The server or the application calculates the index of difficulty of the selected route and compare it with the user profile to provide a customized assessment of the level of physical demand required to face the route. Then, the information appears on the display of the device in use.

1. A method of predicting the a level of physical demand required of an individual to face a given path, comprising the steps of:
determining a physical profile of the individual, including an ability to sustain a given effort during physical activity;
determining a degree of difficulty of the path;
comparing said physical profile with said degree of difficulty to determine the level of physical demand required of the individual,
said physical profile being determined by a first synthetic index, said degree of difficulty is determined by a second synthetic index, said effort level being determined by a function relating said indexesto each other.

2. A method as claimed in claim 1, wherein said first synthetic index and said second synthetic index assume values in a same scale and of the same order of magnitude.

3. A method as claimed in claim 2, wherein the level of physical demand is determined according to a difference between said synthetic indexes.

4. A method as claimed in claim 1, wherein said first synthetic index is calculated from a plurality of parameters selected from:
parameters representative of performance capabilities of the individual,
parameters representative of the ability of the individual to adapt to physical activity, and health risk parameters for the individual.

5. A method as claimed in claim 4, wherein said parameters are combined together, with given weights being assigned to each factor based on scientific evidence.

6. A method as claimed in claim 4, wherein said parameters are selected from age, sex, Body Mass Index calculated from weight and height, waist circumference, heartbeat rate at rest, smoking, hours exercise per week, type of sport being practiced, intensity perceived during physical activity.

7. A method as claimed in claim 1, wherein said parameters are assessed by a questionnaire administered to the individual.

8. A method as claimed in claim 1, wherein said second synthetic index is calculated from parameters representative of potential energy expenditure, both total and per time unit required, said parameters being combined together according to thea total load and the point loads in path segments.

9. A method as claimed in claim 8, wherein said second synthetic index is assigned a numerical value based on the total distance, the total elevation gain and the maximum grade of the path.

10. A method as claimed in claim 1, the method being carried out by a program installed on a server with a web interface, or by an application for a smart phone, a tablet or a PC.

11. A method as claimed in claim 1, wherein the given path comprises a bicycle-touring route.
